(19) United States
(12) Patent Application Publication
(10) Pub. No.: US 2024/0371485 A1
(43) Pub. Date: Nov. 7, 2024

(54) INDIVIDUALIZED DIGITAL ASSISTANT, PERFORMANCE TRACKER, AND SUPPORT TOOL FOR INDIVIDUALS WITH DEVELOPMENTAL DISABILITIES

(71) Applicant: Autism Model School, Toledo, OH (US)

(72) Inventors: Joel VIDOVIC, Ann Arbor, MI (US); Alison THOMAS, Toledo, OH (US); Amy TRAUTWEIN, Toledo, OH (US)

(73) Assignees: Autism Model School, Toledo, OH (US); Autism Model School, Toledo, OH (US)

(21) Appl. No.: 18/380,332

(22) Filed: Oct. 16, 2023

Related U.S. Application Data
(60) Provisional application No. 63/463,937, filed on May 4, 2023.

(51) Int. Cl. G16H 20/00; G16H 40/67; G16H 80/00

(52) U.S. Cl. CPC G16H 20/00; G16H 40/67; G16H 80/00

(57) ABSTRACT

Systems and methods are provided including a trainer computing device for a trainer providing training to a trainee having a developmental disability. A server stores data for a task having task steps, each having associated instructions. A trainee computing device outputs the associated instructions for each task step while the trainee is performing the task. The trainer computing device enables the trainer to monitor progress of the trainee and assign a task step score indicating a level of guidance provided by the trainer while the trainee was completing each task step. The server selects a complexity level to be used during a subsequent training session when the trainee performs the task based on the task step scores for the task steps, the complexity level indicating training conditions and an amount of oversight to be provided by the trainer to the trainee during the subsequent training session.

### CROSS-REFERENCE TO RELATED APPLICATIONS

This application claims the benefit of U.S. Provisional Application No. 63/463,937, filed on May 4, 2023. The entire disclosure of the above application is incorporated herein by reference.

### FIELD

The present disclosure relates to digital assistants, performance trackers, and support tools for individuals with development disabilities.

### BACKGROUND

This section provides background information related to the present disclosure which is not necessarily prior art.

Individuals with developmental disabilities, including, for example, autism, behavior disorders, brain injury, cerebral palsy, Down syndrome, fetal alcohol syndrome, intellectual disability, spina bifida, etc., require special care and instruction from direct service providers and teachers in order to learn how to perform various tasks. Prior traditional systems utilize printed binders with step-by-step instructions printed on sheets within a physical binder. The traditional systems, however, are not adaptable and require the direct service provider or teacher to utilize a separate system for tracking notes on the individual's performance.

### SUMMARY

This section provides a general summary of the disclosure and is not a comprehensive disclosure of its full scope or all of its features.

A system is provided and includes a trainer computing device configured for operation by a trainer providing training to a trainee, the trainee having a developmental disability, the trainer computing device being configured for communication with a server that stores data for at least one task having a plurality of task steps, each task step having associated instructions for performing the task step. The system also includes a trainee computing device configured for operation by the trainee, the training computing device being configured for communication with the server and configured to output the associated instructions for each task step of the plurality of task steps while the trainee is performing the at least one task. The trainer computing device enables the trainer to: monitor progress of the trainee operating the trainee computing device while the trainee is performing the plurality of task steps of the at least one task; and assign a task step score from a plurality of task step scores for each task step of the plurality of task steps after the trainee has completed each task step, the task step score indicating a level of guidance provided by the trainer to the trainee while the trainee was completing each task step. The server is configured to select a complexity level from a plurality of complexity levels to be used by the trainer during a subsequent training session when the trainee performs the at least one task based on the task step scores for the plurality of task steps, the complexity level indicating training conditions and an amount of oversight to be provided by the trainer to the trainee during the subsequent training session.

In other features, the associated instructions for each of the plurality of tasks steps output by the trainee computing device to the trainee while the trainee is performing the at least one task include at least one of: video output; audio output; image output; and textual output.

In other features, the plurality of task step scores include an independent score indicating that the trainee was able to independently complete the associated task step without assistance.

In other features, the server is configured to select the complexity level based on a number of task steps from the plurality of task steps that were assigned with the independent score by the trainer.

In other features, the plurality of task step scores additionally include at least one of: a full physical score indicating that the trainer provided full physical assistance to the trainee while the trainee was performing the associated task step; a partial physical score indicating that the trainer provided partial physical assistance to the trainee while the trainee was performing the associated task step; and a gesture score indicating that the trainer provided a gesture to the trainee to assist the trainee while the trainee was performing the associated task step.

In other features, the trainer computing device is configured to display the associated instructions that are also being output to the trainee with the trainee computing device.

In other features, the trainer computing device is configured to direct the trainee computing device, through communication with the server, to move to a different task step within the plurality of task steps based on input received from the trainer with the trainer computing device.

In other features, each of the plurality of complexity levels indicate at least one of: a trainer-to-trainee ratio; whether the trainer is in close proximity to the trainee; whether the trainer is in a room with the trainee; and whether verbal praise is provided after each step.

In other features, the trainee computing device is configured to provide additional instructions to the trainee in response to the trainee encountering a problem during performance of a task step.

In other features, the trainer computing device and the trainee computing device are each one of a laptop, a tablet, and a smartphone.

A method is also provided and includes communicating, using a trainer computing device, with a server that stores data for at least one task having a plurality of task steps, each task step having associated instructions for performing the task step, the trainer computing device being configured for operation by a trainer providing training to a trainee, the trainee having a developmental disability. The method also includes communicating, using a trainee computing device, with the server, the trainee computing device being configured for operation by the trainee and configured to output the associated instructions for each task step of the plurality of task steps while the trainee is performing the at least one task. The method also includes monitoring, with the trainer computing device, progress of the trainee operating the trainee computing device while the trainee is performing the plurality of task steps of the at least one task. The method also includes assigning, with the trainer computing device, a task step score from a plurality of task step scores for each task step of the plurality of task steps after the trainee has completed each task step, the task step score indicating a level of guidance provided by the trainer to the trainee while the trainee was completing each task step. The method also includes determining, with the server, a complexity level from a plurality of complexity levels to be used by the trainer during a subsequent training session when the trainee performs the at least one task based on the task step scores for the plurality of task steps, the complexity level indicating training conditions and an amount of oversight to be provided by the trainer to the trainee during the subsequent training session.

In other features, the associated instructions for each of the plurality of tasks steps output by the trainee computing device to the trainee while the trainee is performing the at least one task include at least one of: video output; audio output; image output; and textual output.

In other features, the plurality of task step scores include an independent score indicating that the trainee was able to independently complete the associated task step without assistance.

In other features, the server is configured to determine the complexity level based on a number of task steps from the plurality of task steps that were assigned with the independent score by the trainer.

In other features, the plurality of task step scores additionally include at least one of: a full physical score indicating that the trainer provided full physical assistance to the trainee while the trainee was performing the associated task step; a partial physical score indicating that the trainer provided partial physical assistance to the trainee while the trainee was performing the associated task step; and a gesture score indicating that the trainer provided a gesture to the trainee to assist the trainee while the trainee was performing the associated task step.

In other features, the method includes displaying, with the trainer computing device, the associated instructions that are also being output to the trainee with the trainee computing device.

In other features, the method includes directing, with the trainer computing device, the trainee computing device, through communication with the server, to move to a different task step within the plurality of task steps based on input received from the trainer with the trainer computing device.

In other features, each of the plurality of complexity levels indicate at least one of: a trainer-to-trainee ratio; whether the trainer is in close proximity to the trainee; whether the trainer is in a room with the trainee; and whether verbal praise is provided after each step.

In other features, the method includes providing, with the trainee computing device, additional instructions to the trainee in response to the trainee encountering a problem during performance of a task step.

In other features, the trainer computing device and the trainee computing device are each one of a laptop, a tablet, and a smartphone.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a block diagram of a system in accordance with the present disclosure.

FIG. 2 is a diagram illustrating a hierarchy of an example program, with modules, tasks, and tasks steps, in accordance with the present disclosure.

FIG. 3 is a diagram illustrating a hierarchy of an example program, with modules, tasks, and tasks steps, in accordance with the present disclosure.

FIGS. 4-20 are screenshots from a system in accordance with the present disclosure.

Corresponding reference numerals indicate corresponding parts throughout the several views of the drawings.

### DETAILED DESCRIPTION

Example embodiments will now be described more fully with reference to the accompanying drawings.

The present disclosure is directed to systems and methods for an individualized digital assistant, performance tracker, and support tool for individuals with developmental disabilities. The systems and methods include and implement a customizable cloud/web-based curriculum and personalized support tool for individuals with a range of developmental disabilities, including, for example, autism, behavior disorders, brain injury, cerebral palsy, Down syndrome, fetal alcohol syndrome, intellectual disability, spina bifida, etc. With the systems and methods of the present disclosure, a direct service provider or teacher can utilize a trainer computing device to monitor and communicate with an individual with a developmental disability that is using a trainee computing device that guides the individual through a series of steps and tasks to perform and complete a particular designated goal, as discussed in further detail below. The trainee computing device can provide step-by-step instructions that include pictures, video, and audio guidance to the individual. If the individual encounters an issue, the individual can request assistance that can be provided to the individual by the trainer utilizing the trainer computing device in real time and/or by additional step-by-step instructions provided to the individual on the trainee computing device. The system can monitor and track the individual's progress and the time and amount of assistance needed for the individual to complete the designated goal and automatically provide grading and scoring so that the individual's progress with completing the designated goal can be tracked and quantified over time and adjustments can be made to the curriculum and assistance provided to the individual to customize and tailor the provided guidance to most effectively and efficiently teach and train the individual to learn and complete the tasks and goals. Additionally, the quantifiable results of the individual's progress provided by the system can be reported to third parties over time to monitor the results of the systems and methods of the present disclosure and gauge the effectiveness of the systems and methods of the present disclosure for purposes of determining funding, insurance coverage and expenditures, etc.

In this way, and as discussed in further detail below, the visual technology provided by the systems and methods of the present disclosure allow direct service providers to break everyday tasks, such as brushing teeth, taking a shower, making a meal, etc., into individual steps that are displayed one-at-a-time on a tablet or mobile device operating by the individual. Each step within an activity can include a picture, video, audio, and/or textual guidance and cues to assist the individual as they use the system to help guide them through completion of the designated tasks and goals. The direct service providers can track, quantify, and report on the level of the individual's performance and the individual's improvement over time.

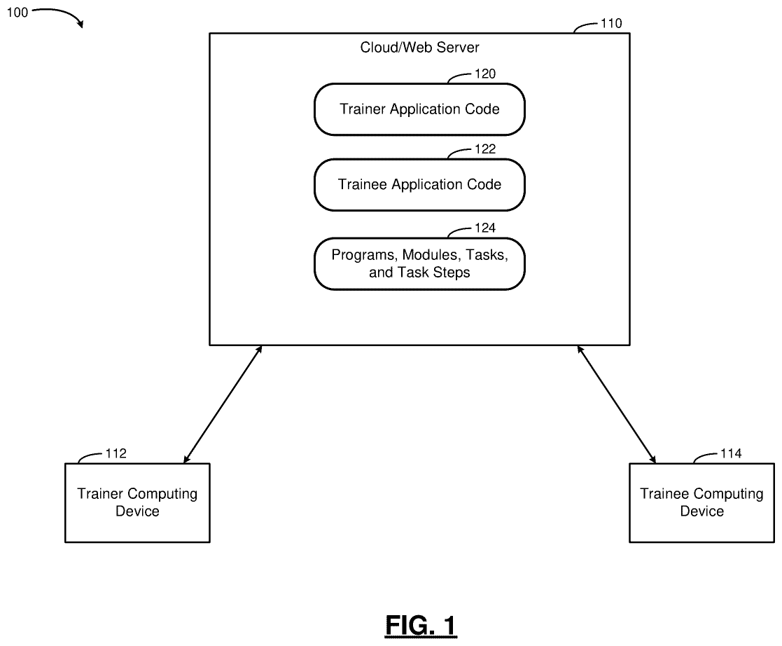

With reference to FIG. 1, a system 100 for an individualized digital assistant, performance tracker, and support tool is illustrated and includes a cloud/web server 110, a trainer computing device 112, and a trainee computing device 114. The trainer computing device 112 and trainee computing device 114 can be implemented by a desktop computer, a laptop, a tablet, a smartphone, mobile computing device, or other computing device configured to perform the described functionality of each device. In one embodiment, the trainee computing device 114 is implemented by a tablet, smartphone, or mobile computing device operated by the individual with the developmental disability while the individual is carrying out and performing a designated task or set of tasks. The trainer and trainee computing devices 112, 114 are each in communication with the cloud/web server 110 that stores and distributes the application code and necessary information and data to the trainer and trainee computing devices 112, 114 to configure the trainer and trainee computing devices 112, 114 to perform the described functionality for each device. For example, the trainer and trainee computing devices 112, 114 can communicate with the cloud/web server 110 over a communication network, such as the Internet, and/or over a local area network (LAN) or a wide area network (WAN). The cloud/web server 110 stores trainer application code 120 that is distributed to and executed by the trainer computing device 112 and trainee application code 122 that is distributed to and executed by the trainee computing device 114. The trainer and trainee application code 120, 122 can include code for standalone applications that are executed by the trainer and trainee computing devices 112, 114, respectively. For example, the code can execute as a desktop application on a desktop computer or laptop. Additionally or alternatively, the trainer and trainee application code 120, 122 can be mobile application code that is downloaded and available from an application store of a smartphone or tablet manufacturer for execution by a smartphone or tablet. In such case, the trainer and trainee application code 120, 122 is stored and made available for download from an application store of the device manufacturer of the smartphone or tablet. Additionally or alternatively, the trainer and trainee application code 120, 122 can be configured as application code that is executed within a web browser running on the trainer and trainee computing devices 112, 114, respectively. While a single cloud/web server 110 is illustrated in FIG. 1, the functionality of the web server 110 can be distributed amongst a number of cloud/web servers cooperatively operating in parallel. In addition, while a single trainer computing device 112 and single trainee computing device 114 are shown, the system 100 can include any number of trainer and trainee computing devices 112, 114.

### Basic Organization of Curriculum

The systems and methods of the present disclosure include a variety of “Activity Schedules” that make up the core curricular content of the program. The content is organized into categories referred to as “Programs,” “Modules,” “Tasks,” and “Task Steps.” Programs include broad categories of skills such as Daily Living Skills, Vocational Skills, Job Training, etc. Modules are sub-categories within a Program and each include a variety of related Tasks. Tasks are specific activities within a Module. Tasks are made up of a variety of individual Task Steps. Task Steps are the individual steps that make up the overall Task. As discussed in further detail below, the system can provide a variety of supporting information and documentation for each of the Tasks Steps within a Task to help guide an individual to complete the Task by focusing on individual Tasks Steps one at a time, with visual cues and guidance. Each Task Step, for example, can include pictures, videos, audio information, and textual instructions and cues to assist the individual to complete the Task.

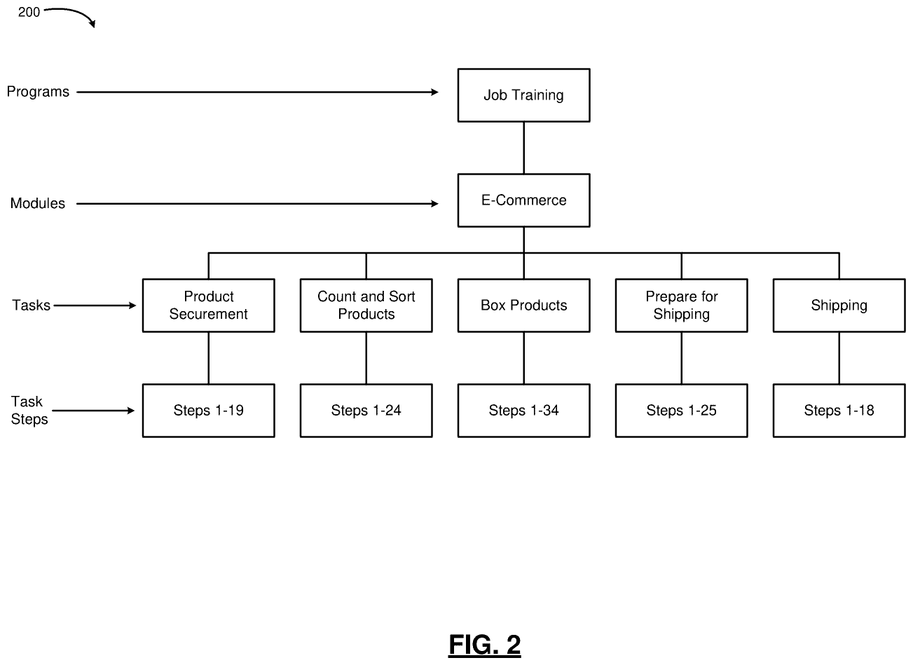

With reference to FIG. 2, a Job Training example Program 200 in accordance with the present disclosure is illustrated. As illustrated in FIG. 2, the Job Training Program includes an E-Commerce Module. The E-Commerce Module includes five Tasks, including Product Securement, Count and Sort Products, Box Products, Prepare for Shipping, and Shipping. Each of the Tasks include a number of Task Steps. For example, the Product Securement Task includes 19 Task Steps, the Count and Sort Products Task includes 24 Task Steps, and so on.

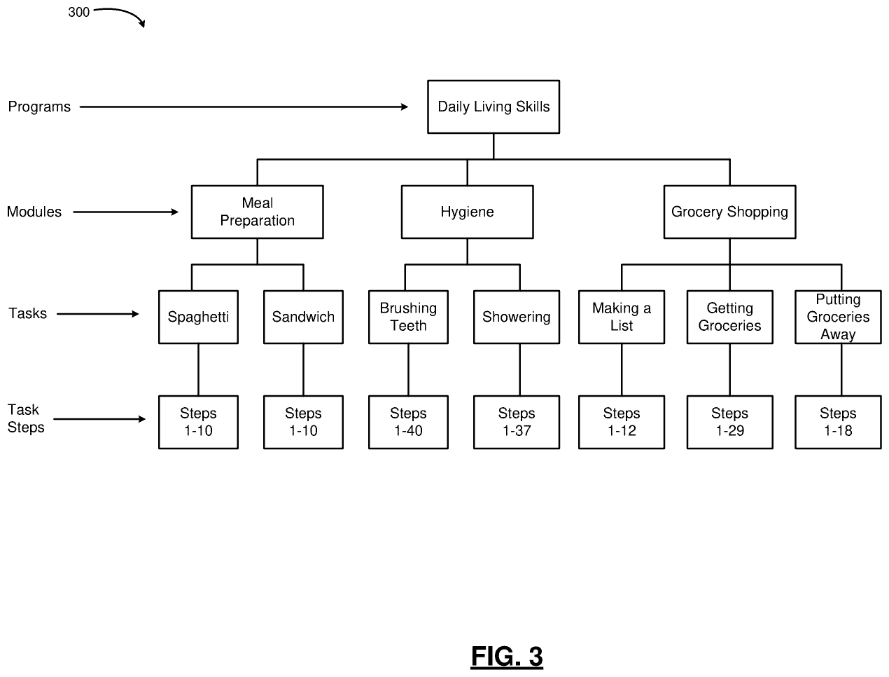

With reference to FIG. 3, a Daily Living Skills example Program 300 in accordance with the present disclosure is illustrated. The Daily Living Skills includes three modules, including a Meal Preparation Module, a Hygiene Module, and a Grocery Shopping Module. Each of the Modules includes a number of Tasks. For example, the Meal Preparation Module includes a Spaghetti Task for preparing spaghetti and a Sandwich Task for preparing a sandwich. The Hygiene Module includes a Brushing Teeth Task and a Showering Task. The Grocery Shopping Module includes a Making a List Task, a Getting Groceries Task, and a Putting Groceries Away Task. Each of the Tasks includes a number of Task Steps. For example, the Spaghetti Task includes 10 Task Steps, the Sandwich Task includes 10 Task Steps, the Brushing Teeth Task includes 40 Task Steps, and so on.

### Trainer Computing Device and Interface

This section discusses and describes the functionality performed and carried out by users having a trainer role while operating and interacting with a trainer computing device 112. Users having the trainer role can include, for example, direct service providers in charge of teaching and training assigned individuals having developmental disabilities. For example, users having a trainer role are able to perform the following activities while operating and interacting with a trainer computing device 112: initiate and conduct training sessions with assigned trainees; customize training content for trainees; view past evaluations for assigned trainees; view a variety of evaluations, scoring, and reports for assigned trainees. As discussed in further detail below, the systems and methods of the present disclosure provide evaluations, scoring, and reports that enable instantaneous analysis of data collected during training sessions. In addition, the systems and methods of the present disclosure enable the trainer to embed problem solving opportunities within a training session. Prior paper-based activity schedule systems, for example, are not able to seamlessly provide guidance for a trainee when a problem or issue arises while the trainee is performing a given Task or Task Step. With the systems and methods of the present disclosure, trainers can conceptualize and anticipate the types of problems that may arise during a given Task or Task Step sequence and enable customized step-by-step solutions that can be triggered to assist the trainee in solving the problem independently during the training.

The user initially logs into the system with a trainer computing device 112 by, for example, entering the user's associated login credentials, such as an email and password, into the application's login screen. Once the user is authenticated as a trainer, the cloud/web server 110 provides a trainer dashboard to the trainer computing device 112 for display to the user/trainer. The trainer dashboard can include a list of particular trainees that have been assigned to the particular trainer. For example, the trainer dashboard can include a dropdown menu that includes the names of the particular trainees that have been assigned to the trainer.

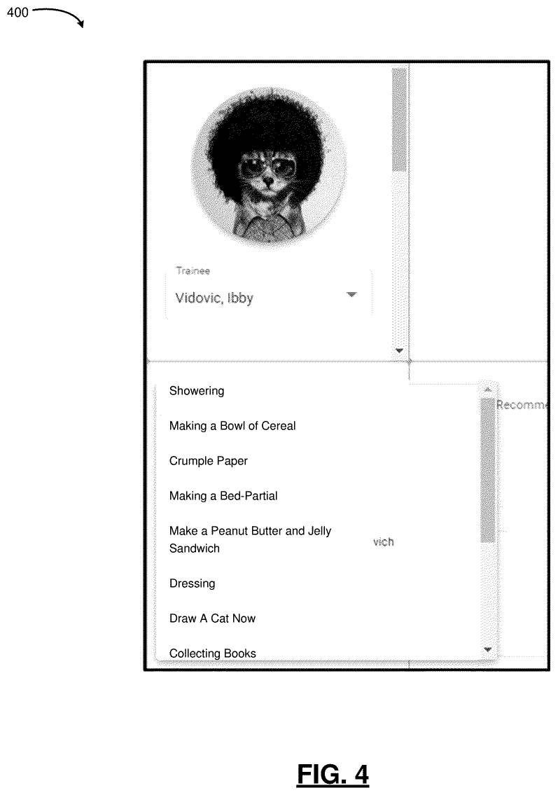

With reference to FIG. 4, a screenshot 400 of the trainer dashboard is shown wherein the Trainee “Ibby Vidovic” has been selected and a list of associated Tasks available for selection are shown, including: showering, making a bowl of cereal, crumple paper, making a bed-partial, make a peanut butter and jelly sandwich, dressing, draw a cat now, and collecting books. The trainer can select a particular Task from the available list of Tasks.

Once a Task is selected, additional information is provided regarding the last time the trainee participated in a training session for that Task. The system automatically generates a recommended complexity level for the Task based on the results of the trainee's prior performance of the task. The complexity levels refer to the conditions in which the training will occur and the amount of guidance or oversight to be provided by the trainer during the training session. For example, the systems and methods of the present disclosure can include six complexity levels, as follows, with the noted associated training conditions:
Complexity Level 1: setting with 1 to 1 trainer-to-trainee ratio; instructor is in close proximity; verbal praise/feedback is provided after each step; continue until mastery criteria reached.
Complexity Level 2: setting with 1 to 3 trainer-to-trainee ratio; instructor is in the room; verbal praise/feedback provided occasionally throughout task completion; continue until mastery criteria reached or maintained.
Complexity Level 3: setting with 1 to 3 trainer-to-trainee ratio; instructor is in the room; verbal praise/feedback provided occasionally throughout job completion; introduce problem solving skills; sabotage job; and/or missing materials.
Complexity Level 4: setting with 1 to 3 trainer-to-trainee ratio; instructor is out of the room; all visuals; problem solving included occasionally; verbal praise/feedback provided ONLY after job completion and supervisor notification is complete.
Complexity Level 5: setting with 1 to 3 trainer-to-trainee ratio; instructor is out of the room; all visuals; problem solving included occasionally; verbal praise/feedback provided ONLY after job completion and supervisor notification is complete; increased emphasis on how to interact with peers and have fun during work.
Complexity Level 6: setting with 1 to 3 trainer-to-trainee ratio; instructor is out of the room; visuals available as needed; problem solving included occasionally; verbal praise/feedback provided ONLY after job completion and supervisor notification is complete.

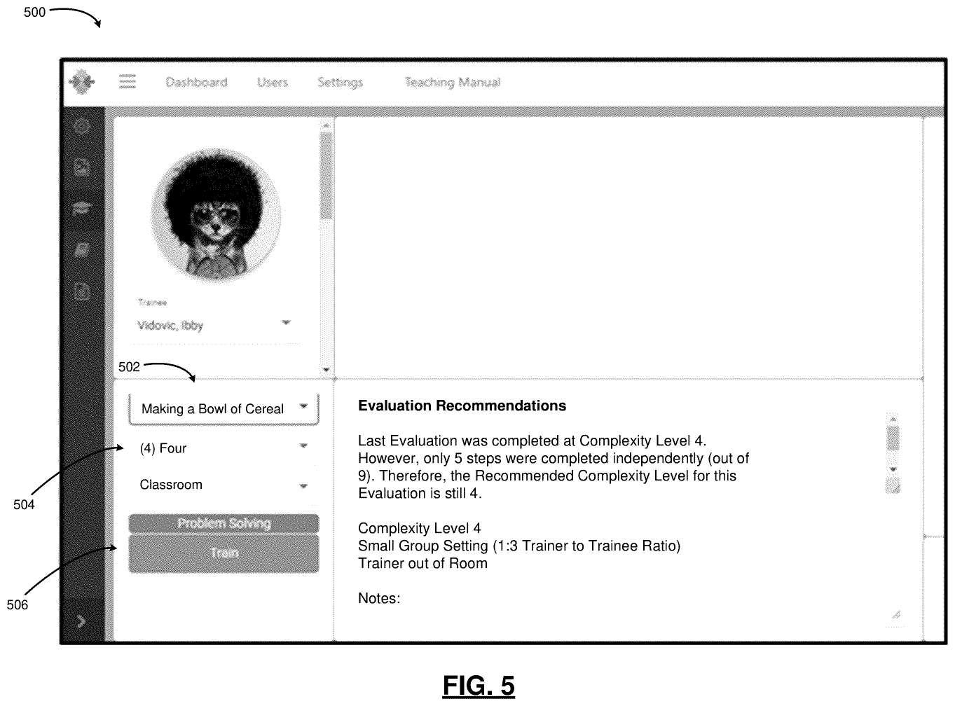

With reference to FIG. 5, a screenshot 500 of the trainer dashboard is shown wherein the Task for “Making a Bowl of Cereal” has been selected at 502. As shown, the Evaluation Recommendations indicate that the last time this Task was performed, it was completed at complexity level 4, but that only 5 of the 9 steps were completed independently. As such, the system provides a recommended complexity level that remains at complexity level 4 for the evaluation of this Task. As shown at 504, complexity level 4 is automatically selected based on the recommendation. The trainer, however, can review the recommended complexity level and manually select a different complexity level. Once the complexity level is selected, the trainer can choose to being a training session, or activate a problem solving scenario for the training session.

In this way, the system automatically reviews and evaluates the trainee's previous performance of the Task and determines whether to increase or decrease the complexity level based on the trainee's previous performance. For example, if the percentage of Task Steps performed independently is greater than a threshold percentage, such as 75%, the system can recommend that the complexity level be incremented to the next highest complexity level. Additionally, if the percentage of Task Steps performed independently is less than a threshold percentage, such as 25%, the system can recommend that the complexity level be decreased to the next lowest complexity level. Based on the complexity level recommended by the system, the trainer can then monitor and administer the training session for performing the current Task and set of Task Steps with appropriate guidance in accordance with the recommended complexity level.

### Conducting a Training Session Without Problem Solving

Once the trainer has selected the Task and complexity level, the trainer can activate the training session by selecting the “Train” button 506. Selecting the “Train” button will initiate a training session for the selected Task on both the trainer and trainee computing devices 112, 114. As discussed below, the selected trainee, e.g., the selected individual with a developmental disability, must be logged into a corresponding trainee computing device 114 in order for the training session to begin.

Once the training session is activated, the trainee can view the first step in the assigned task on the trainee computing device 114, while the trainer is able to see a list of all steps included in the task. The trainer and trainee can participate in the training session while near each other and in the same room, or while remote from each other with the trainer monitoring the trainee's progress with the trainer computing device 112. The trainer's location can depend, for example, on the selected complexity level.

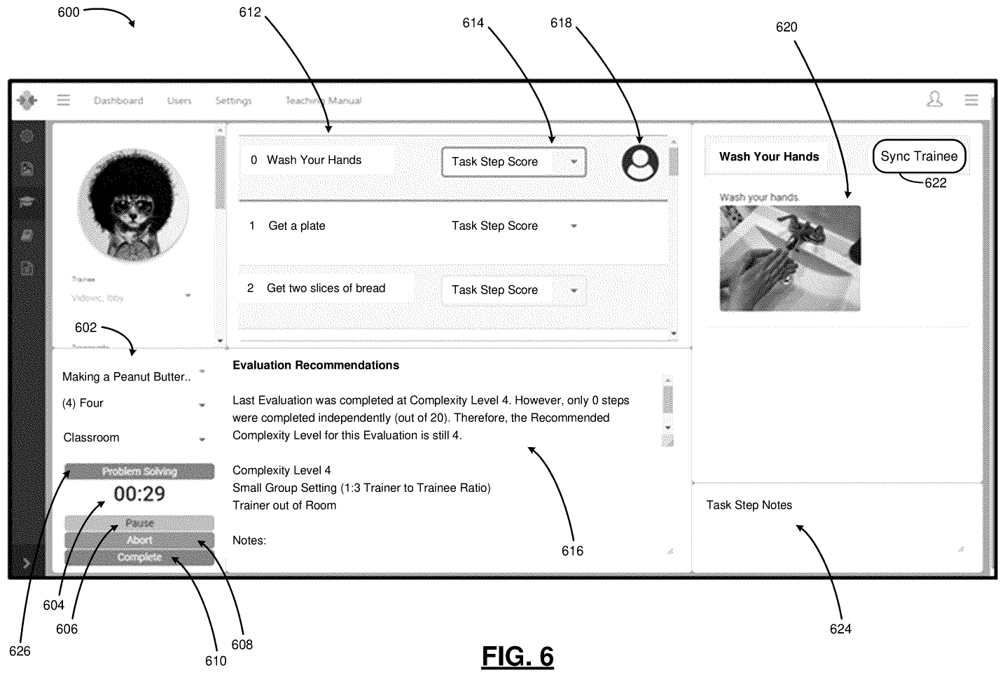

With reference to FIG. 6, a screenshot 600 of the trainer interface as displayed on the trainer computing device 112 is shown wherein the Task for “Making a Peanut Butter and Jelly Sandwich” has been selected at 602. As shown at 604, a digital timer automatically begins when the training session is started in order to track the length of time it takes for the trainee to complete the task. The interface also includes a pause button 606 to pause the training session, an abort button 608 to terminate the training session, and a complete button 610 to designate the training session and the associated Task as being completed.

The interface also includes a panel 612 that displays a list for each Task Step within the designated Task. For example, the initial Task Steps shown in FIG. 6 include “Wash Your Hands,” “Get a plate,” “Get two slices of bread,” etc. Each Task Step includes an associated “Task Step Score” dropdown box that allows the trainer to score the trainee's level of independence in performing the associated Task Step. For example, the Task Step Score can indicate whether the trainee was able to independently complete the Task Step or, if assistance was required, the level of assistance provided by the Trainer. For example, the Task Step Score drop down can include scoring options to indicate “Full Physical,” “Partial Physical,” “Gesture,” and “Independent.” The trainer can select Independent if the trainee was able to independently complete the Task step without assistance. The trainer can select Full Physical or Partial Physical when the trainee required full physical or partial physical assistance to complete the Task Step. The trainer can select Gesture to indicate that the trainer provided gesture guidance, such as a hand gesture or indication, without full or partial physical assistance. While these Task Step Scores are provided as examples for the illustrated Task Step, other scoring systems with additional or fewer indicators for assistance can be utilized in accordance with the present disclosure.

At 616, information about the trainee's most recent training session for the particular Task is illustrated. In this way, the trainer has immediate access to information about the trainee's previous performance. The trainer can also enter notes about the training session as a whole for storage and future reference. For example, the trainer can provide notes indicating important information, such as a step in the Task that needs to be revised or that the trainee appeared tired on the particular day of the training, such that performance may have been impacted.

An icon 618 is used to indicate to the trainer the particular Task Step that the trainee is currently viewing on the trainee computing device 114. This feature allows the trainer to monitor the training session from a distance while remote, such as in a different room, from the trainee. With previous traditional paper-based systems, the trainer would continually need to approach the trainee and hover near the trainee in order to determine which step the trainee was currently viewing and working on. The hovering and proximity of the trainer, however, can interfere with the trainee's performance and increase anxiety for trainees as they feel increased monitoring of their performance. As the trainee moves through the Task Steps of the Task, the icon 618 on the trainer's interface moves down the list of Task Steps.

At 620, the current support being displayed to the trainee is shown. For example, a graphic, video, text, or a link to audio support can be illustrated at 620 to show the trainer the type of supporting guidance that is currently being displayed to the trainee for the particular Task Step.

A “Sync Trainee” button 622 is provided that enables the trainer to remotely set the trainee's interface screen on the trainee computing device 114 to a particular Task Step. For example, if the trainer notices that the trainee has accidentally skipped a Task Step in the Task, the trainee can use the Sync Trainee button 622 to set the trainee's interface back to the missed Task Step so that a correction can be made and the skipped Task Step can be performed. Importantly, the trainer is able to control the trainee's interface on the trainee computing device 114 remotely and without having to physically approach the trainee while the trainee is performing the Task Step, which could increase anxiety of the trainee and negatively impact performance of the Task.

At 624 of the trainer interface, a text entry box is provided so that the trainer can make specific notes about a particular Task Step within the Task. For example, if the trainer noticed that the trainee had difficulty with the fine motor skills necessary to open a jar on a particular Task Step, the trainer can make a note at 624 that some adaptive equipment could be explored to help the trainee complete the step with more independence next time.

Once all of the Task Steps have been completed, the trainer selects the Complete button 610 to indicate that the Task has been completed.

### Conducting a Training Session With Problem Solving

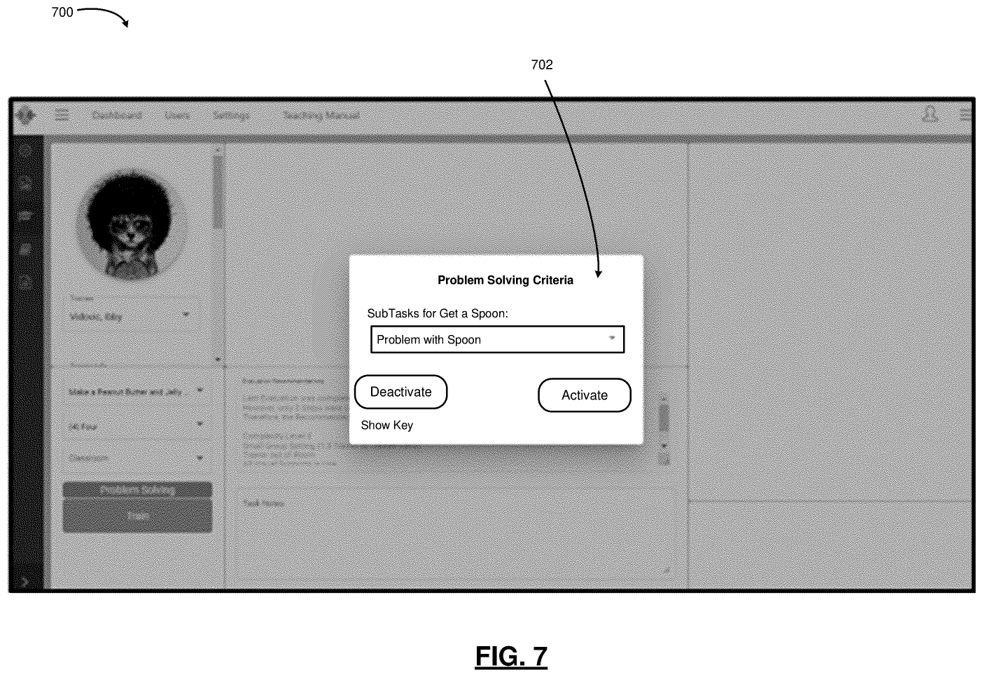

When setting up a training session, the trainer can initiate a problem-solving lesson by selecting the problem-solving button 626. Selection of the problem-solving button 626 opens a new menu where all available problem-solving scenarios for the selected Taks can be viewed, as shown in FIG. 7 at 702. Once a problem-solving scenario has been activated, the trainer can begin the training session and the new problem solving sub steps will be added to any task for which problem solving was activated.

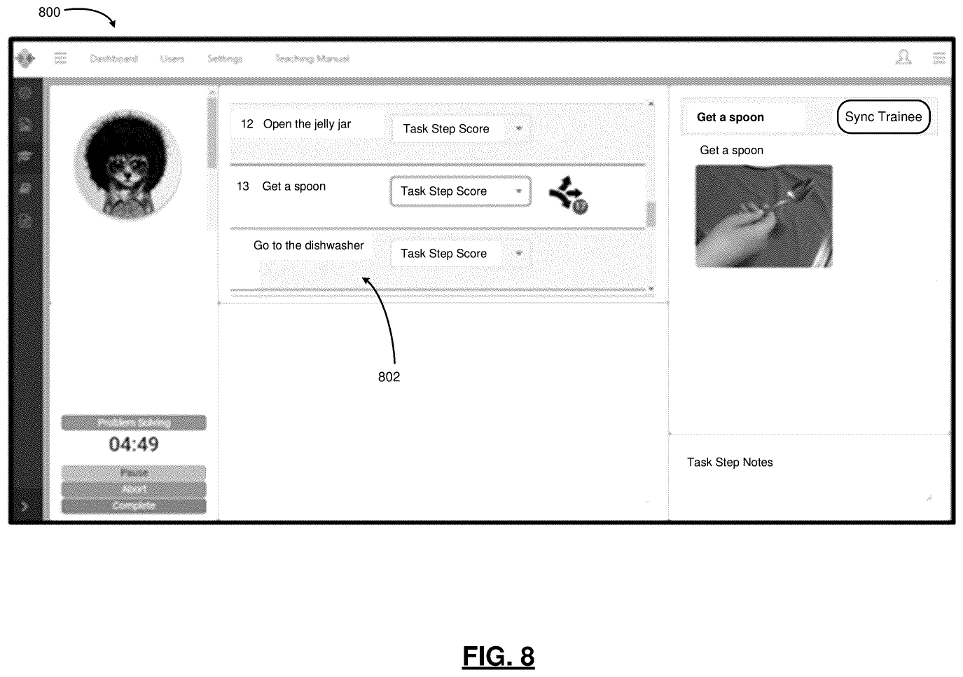

With reference to FIG. 8, the trainer interface is shown with a problem-solving sub step 802 added to Step 13 for the Task Step of “Get a spoon.” In this case, for example, the trainee encountered a situation where a spoon was not available in the silverware drawer. Problem solving was then initiated to add a problem-solving sub step to direct the trainee to the dishwasher to find the missing spoon still in the dishwasher after being washed. In this way, the trainee is able to problem solve the encountered problem of a missing spoon from the drawer.

Problem solving sub steps are customizable so that any problem can have a uniquely generated solution.

Customized Training Content with Trainee Specific Overrides

Previous traditional paper-based activity schedules require trainers to frequently reinvent a new activity schedule each time they would like to add a Task for a trainee. With the systems and methods of the present disclosure, however, trainers have access to an entire library of tasks that can be easily shared and assigned to any trainee with whom they are working.

In addition, shared tasks can be customized for individual trainees by implementing a trainee specific task override, which ensures that all Tasks remain individually tailored to the unique needs of the individual trainees. For example, trainers can use the trainee specific task override feature to: hide/delete a Task Step within a Task; replace a Task Step within the Task with a different Task Step; and insert an additional Task Step into the Task. For example, the trainer can view all current Task Steps associated with a particular Task and can modify the Task to add a Task Step, replace a Task Step, hide a Task Step, etc. These changes will only affect the chosen task for the selected trainee and the Taks will remain unchanged for all other users.

### Mobile Training Application

While FIGS. 4-8 illustrate example screenshots of a desktop-type application executing on a trainer computing device 112, such as a desktop computer or laptop, the same functionality can be implemented within a mobile application executed on a trainer computing device 112, such as a smartphone or tablet. In many cases, the training experience can be a hands-on type of activity requiring trainers to be in the same room and fully engaged with the trainee during the training session. In such cases, the use of a mobile application version of the training application code 120 with a trainer computing device 112, such as a smartphone or tablet, allows the trainer the greatest flexibility to monitor and collect performance data and input evaluation and scoring data into the system.

For similar reasons, the trainee application code 122 can also be implemented using a mobile application executed on a trainee computing device 114, such as a smartphone or tablet operated by the trainee.

### Trainee Computing Device and Interface

This section discusses and describes the functionality performed and carried out by individuals having a trainee role while operating and interacting with a trainee computing device 114. An individual or trainee accesses the system and methods of the present disclosure by logging into a trainee computing device 114 with login credentials, such as an email and password. The system authenticates and validates the user as a trainee and displays a trainee dashboard showing the trainee's options for training sessions.

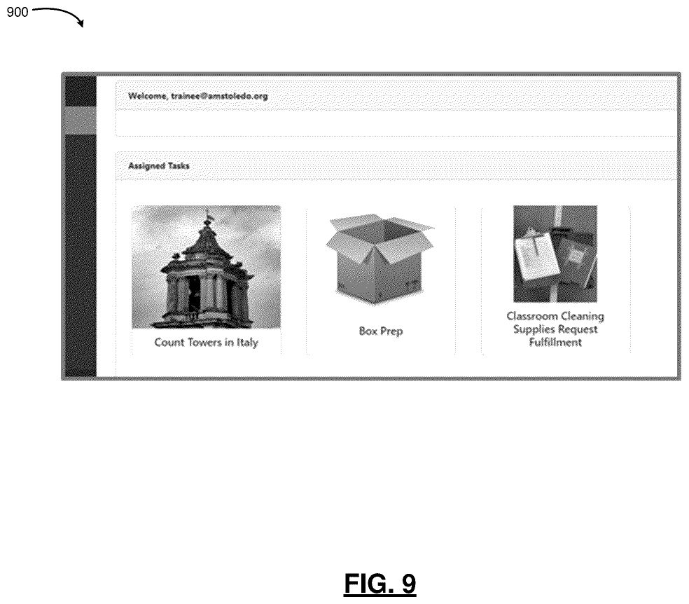

With reference to FIG. 9, a screenshot 900 of a trainee welcome screen is shown illustrating that the particular trainee has been assigned three tasks titled: Count Towers in Italy; Box Prep; and Classroom Cleaning Supplies Request Fulfillment. Each of these tasks would have been previously assigned to the trainee by a trainer using a trainer computing device 112 and includes a set of Task Steps, as discussed above. The trainee can select one of the assigned tasks by clicking on the associated icon for the task.

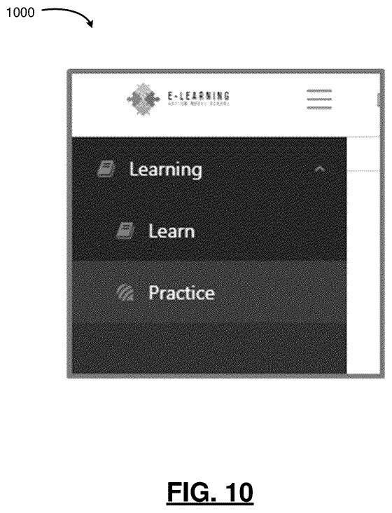

With reference to FIG. 10, the trainee can select to perform the selected assigned task in either a Learning mode or a Practice mode by selecting either “Learn” or “Practice,” respectively, from the displayed menu. In each case, the trainee application guides the trainee step-by-step through the Task Steps necessary to complete the assigned task. Each step can include visual information and cues, such as videos, audio data, text data, pictures, etc., to help guide the trainee through each Task Step necessary to complete the selected assigned task.

### Practice Mode

In practice mode, the trainee is free to advance through the Task Steps as needed without the training session being timed and without performance data being collected about the trainee's performance of the tasks. In other words, the trainee can use the practice mode to walk through and practice the various Task Steps without the results being tabulated, stored, or reported.

### Learn Mode

In learn mode, a trainer actively monitors and scores the trainee's performance of the various Task Steps, as discussed above. In addition, the training session is timed in learn mode and all results, scoring, evaluation and notes of the trainer are stored and reported.

### Problem Solving

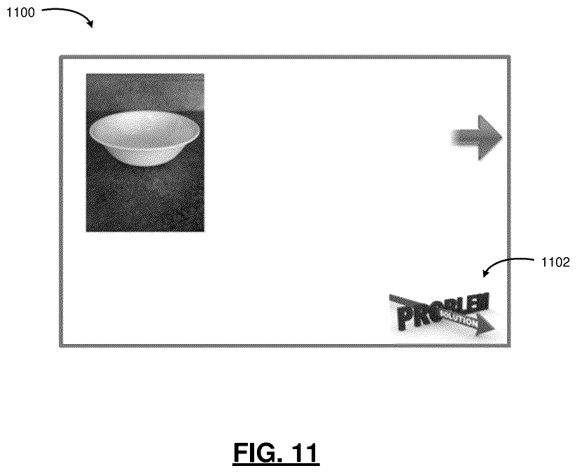

As discussed above, a problem-solving mode can be used when a trainee encounters a problem or issue with respect to the current task. For example, with reference to FIG. 11, a screenshot is shown for the Task Step “Get a bowl from the cupboard,” which is included in a “Make a Bowl of Cereal” Task. In the event the trainee looks in the cupboard and does not find a bowl, the trainee can click the problem-solving icon 1102 to enter the problem solving mode.

In such case, the trainee is presented with a series of problem-solving Task Steps to instead retrieve the bowl from the dishwasher. The Tasks Steps in the problem-solving mode can include, for example: Sub-Task 1—Go to the dishwasher; Sub-Task 2—Open the dishwasher; Sub-Task 3—Get a bowl; and so on.

The seamless presentation of problem-solving sub-tasks and solutions allows trainees to appropriately learn adaptive behavior to solve problems with a given task as they arise and without direct or explicit instructions from the trainer.

### Alternate Steps

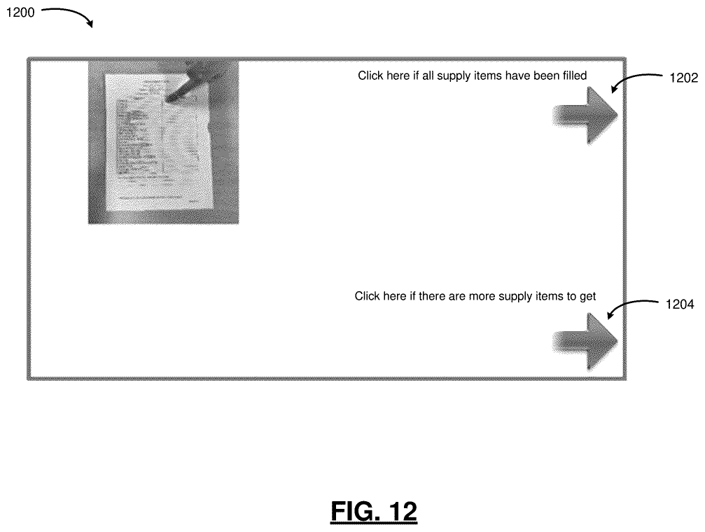

The trainee application allows for the training session to include alternate steps whereby the trainee must select between alternative next steps and then be diverted to different resulting Task Steps depending on the selection. For example, with reference to FIG. 12 a screenshot 1200 is illustrated for a Task of gathering supply items from a supply item list. The illustrated screenshot includes alternative steps whereby the trainee is requested to select a first icon 1202 if all supply items have been filled and to select a second icon 1204 if there are more supply items to collect. If there are more supply items to collect, the training session loops back to a previous Task Step to collect more supply items and continues in this fashion until the trainee selects icon 1202 indicating that all supply items have been filled.

### Portability and Variety of Trainee Supports

As a cloud-based software platform, the systems and methods of the present disclosure enable trainees to access an entire library of mixed media task supports anywhere through the use of a connected device, such as a tablet, smartphone, or laptop. As compared with prior paper-based systems, the systems and methods of the present disclosure enable the user to travel to various locations within a learning environment and perform training using the single trainee computing device 114.

### Scoring, Reporting, and Evaluations

After a training session is completed, the evaluation information about the trainee's performance of the task is transmitted to the cloud/web server 110 for storage. Trainers or other administrators can then access the past evaluations of a particular trainee to view the progress of the trainee over time. The past evaluation data includes the date, time, trainer, trainee, and task that was evaluated. The past evaluations can be searched by trainee, by task, by date range, etc. Trainers and administrators can also edit, delete, or print past evaluations, as needed.

Data collected during a training session can be instantaneously analyzed by the system to provide a variety of important information to help monitor progress of their trainees and make changes to training programs that are based on data from individual trainees. For example, as discussed above, the previous evaluation data can be used by the system to select a recommended complexity level for the trainer to use for guidance during the next training session that the trainee participates in for a particular Task.

### Progress Reports

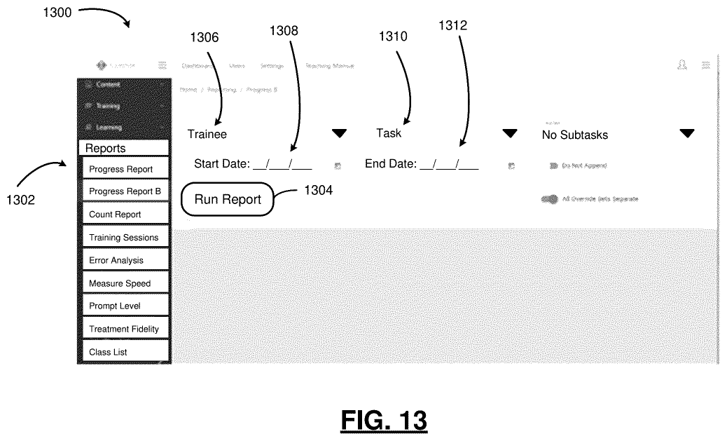

With reference to FIG. 13, a screenshot 1300 is shown illustrating a list of reports 1302 available for selection. The reports including, for example, a Progress Report, a Progress Report B, a Count Report, a Training Session Report, an Error Analysis Report, a Measure Speed Report, a Prompt Level Report, a Treatment Fidelity Report, and a Class List Report. A particular trainee for the report can be selected at 1306 and a particular task for the report can be selected at 1310. A start date and an end date for the report can be entered at 1308 and 1312. Once the various parameters for the report are entered, the report can be run by clicking the Run Report button 1304.

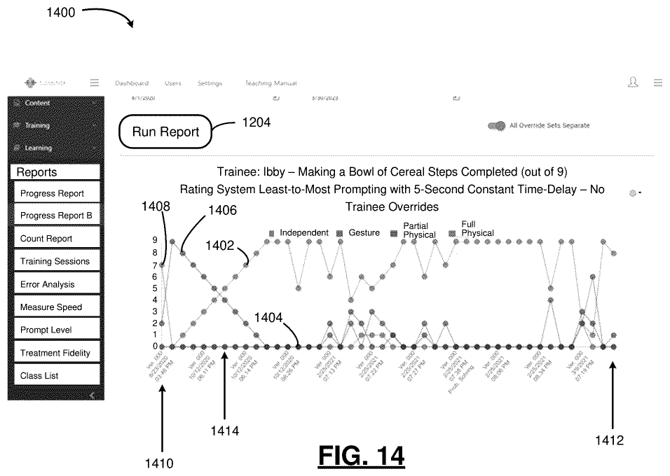

With reference to FIG. 14, a screenshot 1400 is shown of a graph for a report illustrating the progress of a Trainee in completing the Task of Making a Bowl of Cereal. In the graph of FIG. 14, the vertical y-axis represents the number of Task Steps included in the Task. As shown in FIG. 14, there are 9 Task Steps included in the Task of Making a Bowl of Cereal. Individual training sessions for the Task are represented along the horizontal axis, with the progression of time represented from left to right. For example, the trainee's earliest training session for the task is represented in the leftmost column of the graph at 1410 and the trainee's most recent training session for the task is represented in the rightmost column of the graph at 1412. The graph includes four graph lines (1402, 1404, 1406, and 1408) corresponding to the number of Tasks Steps that were completed during a training session at each of four independence scoring levels: Independent; Gesture; Partial Physical; and Full Physical. As discussed above, during a training session, the trainer selects one of the levels to indicate the amount of guidance/instruction provided to the trainee for the given Task Step. Selection of “Independent” represents that the trainee completed the Task Step without instruction or guidance from the trainer. “Gesture” indicates that the trainer provided gesture guidance, such as a hand gesture or indication, without full or partial physical assistance. “Full Physical” or “Partial Physical” indicates that the trainer provided either Full Physical or Partial Physical assistance to the trainee to complete the Task Step. A given column of the graph represents an individual training session. In the graph of FIG. 14, graph line 1402 represents Task Steps that were designated as “Independent.” Graph line 1404 represents Task Steps that were completed as “Gesture.” Graph line 1406 represents Task Steps that were completed as “Partial Physical.” Graph line 1408 represents Task Steps that were completed as “Full Physical.” Because there are nine total Task Steps in the Task of Making a Bowl of Cereal, the number of Tasks Steps for each vertical column add up to nine for the given training session. For example, in the earliest training session shown at 1410, two Task Steps were designated as “Partial Physical” while seven Task Steps were designated as “Full Physical.” In the most recent training session shown at 1412, one Task Step was designated as “Partial Physical” while eight Task Steps were designated as “Independent.” With reference to the training session shown at 1414, four Task Steps were designated as “Partial Physical” and five Task Steps were designated as “Independent.” In this way, the trainee's progress with a given Task can be graphically illustrated, evaluated, and monitored over time to confirm that the trainee is progressing and performing the given Task more independently with less and less guidance and instruction over time.

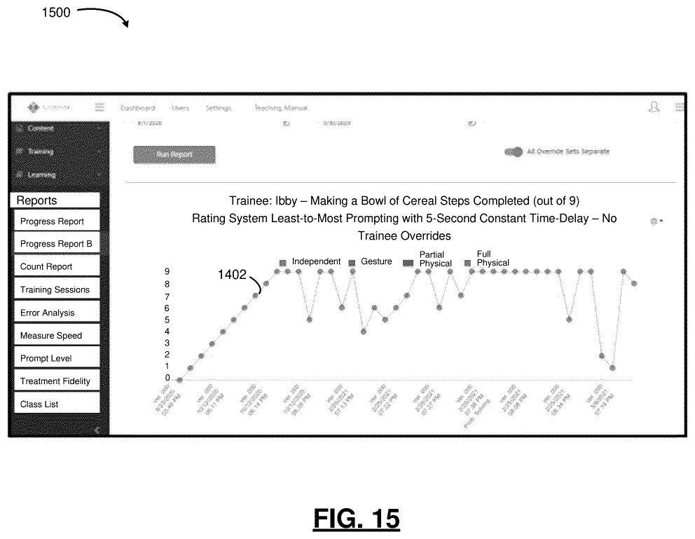

While all four graph lines 1402, 1404, 1406, 1408 are illustrated in FIG. 14, the user can also click on the legend keys representing each of the graph lines 1402, 1404, 1406, 1408 to view any one of the graph lines 1402, 1404, 1406, 1408 in isolation without displaying the other non-selected graph lines. For example, with reference to FIG. 15, a screenshot 1500 is shown with only the graph line 1402 corresponding to “Independent” being shown.

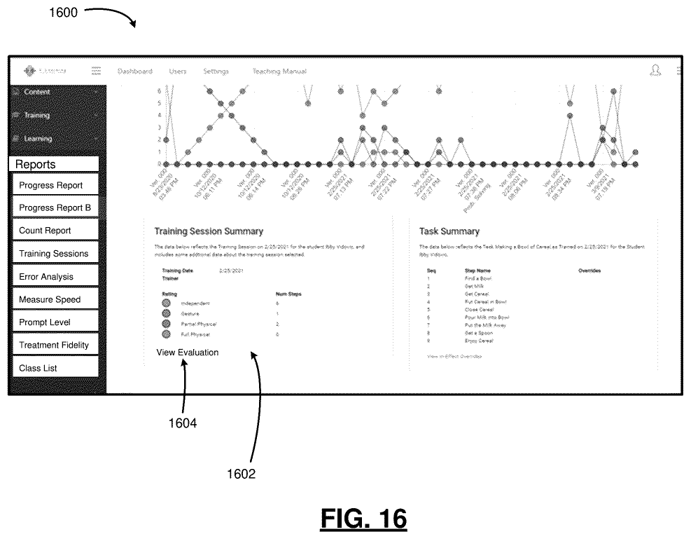

Clicking on any data point within the graph generates a table at the bottom of the screen to enable trainers to quantify the number of steps completed at each level. With reference to FIG. 16, a Training Session Summary 1602 is shown and illustrates the number of steps completed at each designated level for a given training session. In addition, a “view evaluation” link 1604 is provided that enables the user to view a specific evaluation and see the particular Task Steps within a Task that were completed independently, or which particular Task Steps within the Task the trainee needed the most assistance with.

### Training Sessions Report

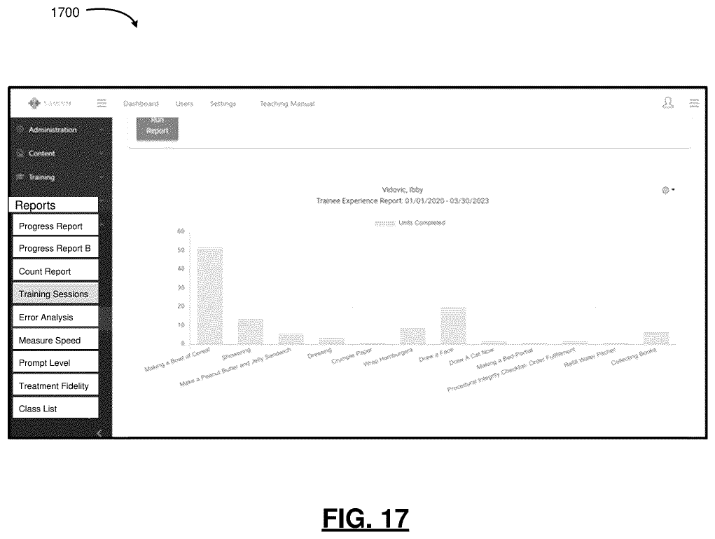

The training session report allows trainers to evaluate how many training sessions a trainee has participated in for each assigned Task during a specified date range. The report enables trainers to monitor whether a trainee is getting sufficient practice for assigned Tasks. A user can, for example, select a particular trainee and date range for the report. As shown in FIG. 17, a screen shot 1700 is shown illustrating a graph showing how many training sessions have been conducted for each task.

### Error Analysis Report

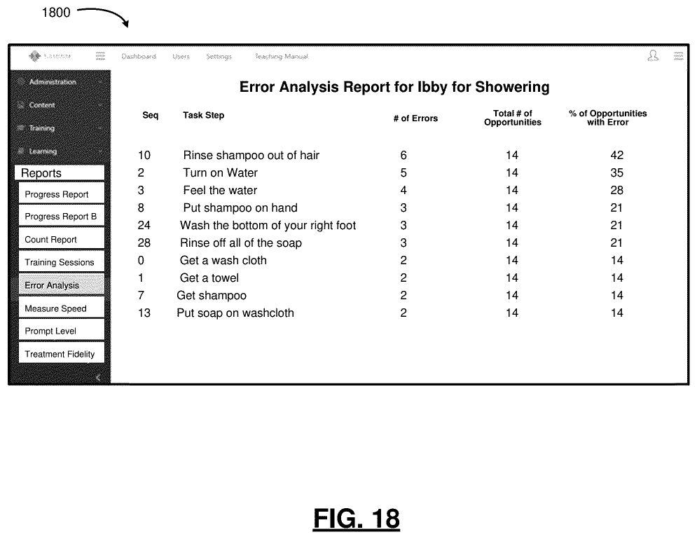

With reference to FIG. 18, a screenshot 1800 of an Error Analysis Report is shown. The Error Analysis Report enables trainers to review performance data from trainees with a specific focus on which particular steps within a task the Trainee most frequently needed help to complete. To run the report, the user selects a task, a trainee, a date range, and a minimum number of training sessions that must have been completed in order to generate the report. The minimum number of training sessions is customizable, as some users may want to ensure an adequate number of training sessions have been completed prior to analyzing data for patterns in errors. The system then analyzes each individual Task Step within the selected Task that occurred during training sessions conducted within the selected date range and identifies how many times each step within the Task was scored as anything other than “Independent.”

The Error Analysis Report then displays a rank order of the Task Steps to identify which Task Steps within the Task the trainee most frequently required help with. The Error Analysis Report can assist trainers to identify task steps that may need to be modified or steps in which additional adaptive equipment may be beneficial for improving independence. As shown in FIG. 18, the selected trainee participated in 14 training sessions focused on showering during the selected timeframe. The Task Step within the showering task that was not independent most frequently was rinsing shampoo out of hair. This particular Task Step was not completed independently in 6 out of the 14 training sessions, or 42% of the time. A trainer can use this information to examine additional supports that could be added to the Task Step to better assist the trainee in becoming more independent with rinsing shampoo out of trainee's hair.

### Measure Speed Report

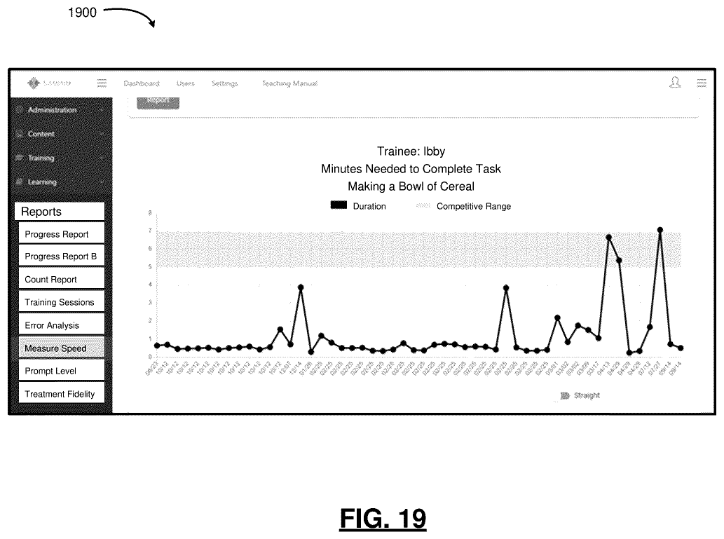

With reference to FIG. 19, a screen shot 1900 of a Measure Speed Report is shown. The Measure Speed Report enables trainers to view and determine how quickly a Trainee can complete a given Task. This can be an important measure of proficiency when evaluating, for example, particular employment related tasks. As shown in FIG. 19, trainers can evaluate how quickly a trainee is able to complete a task and compare the trainee's performance to a designated competitive range. In the example of FIG. 19, the designated competitive range or timeframe to complete the Task of Making a Bowl of Cereal is provided as five to seven minutes.

To run the Measure Speed Report, the trainer selects a trainee, a Task, and a specific date range to evaluate. Trainers can also enter in a customized “minimum competitive range” and a “maximum competitive range” so that the trainee's performance can be viewed in relation to a specific target speed.

### Prompt Level Report

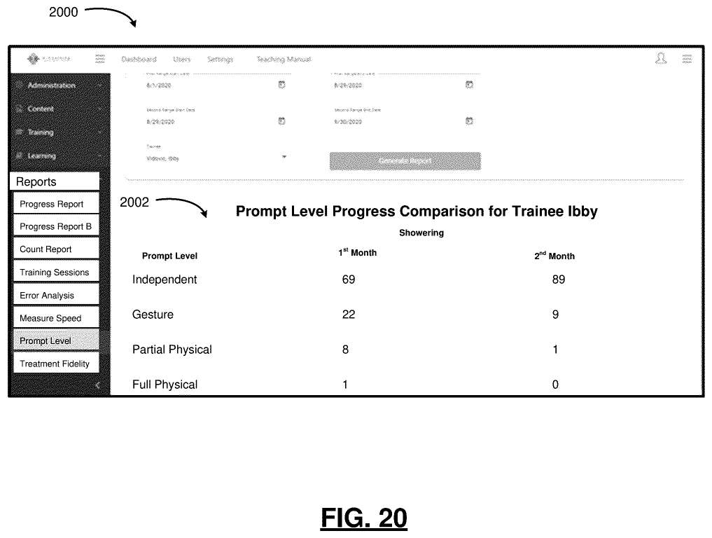

With reference to FIG. 20, a screen shot 2000 of a Prompt Level Report is shown. The Prompt Level Report illustrates the percentage of Task Steps completed at each prompt level for two different time periods. For example, in FIG. 20, a comparison is shown between a first month and a second month.

For some trainees, progress towards independence may move more slowly and be difficult to detect when looking at the progress report and focusing solely on the number of steps completed independently within a given Task. For some trainees, progress may be detected by evaluating whether the trainee is learning to complete the Task Step with less intrusive support and explicit guidance or instruction from a trainer over time. The Prompt Level Report can enable the user to evaluate this type of progress. For example, the Prompt Level Report allows for a direct comparison of the percentage of Task Steps completed at each prompt level over two different time periods. Trainers can select a trainee, a task, and enter two different date ranges to compare, as shown in FIG. 20.

### User Roles

The systems and methods of the present disclosure can be implemented with designations for different user roles. In one example, an implementation can include eight different user roles: System Administrator; Organization Administrator; Division Administrator; Supervisor; Content Editor; Reporting; Trainer; and Trainee. The following Table 1 illustrates the functionality and menus accessible to users within each user role category:

TABLE 1

| | User Role | | | | | | | |
|---|---|---|---|---|---|---|---|---|
| | Sys | Org | Div | | Cont | | | |
| Menu | Admin | Admin | Admin | Supervisor | Ed | Reporting | Trainer | Trainee |
| Sys Admin | ✓ | | | | | | | |
| Administratio | ✓ | ✓ | ✓ | ✓ | | | | |
| Content | ✓ | | | ✓ | ✓ | | | |
| Training | | | | ✓ | | | ✓ | |
| Learning | | | | | | | | ✓ |
| Reports | | | | ✓ | | ✓ | ✓ | |

### Creating Customized Curriculum Content

Some programs, modules, tasks, and task steps may be provided to users within a library of Tasks and Task Steps stored in the cloud/web server 110 with the programs, modules, tasks, and task steps 124. Users can also create their own completely customized activity schedules using various forms of media, including pictures, video, and audio to support the independence of their clients and students, i.e., trainees. For example, media can be selected from a media library stored on the cloud/web server 110 and/or uploaded by the user for subsequent storage in the cloud/web server 110. The ability to create unique curricular content can be provided to those users that have been granted content editor privileges.

The user can assign a learning program name to a new Program to designate how the learning program will be labeled within the Program. The user can also provide a description of the learning Program and a visual cue, such as a picture or image to be associated with the Program. The user can then create new Modules within the Program, new Tasks within each Module, and new Task Steps within each Task. The user can upload and create individual content, instructions, and media to be associated with each Module, Task, and Task Step. For example, the user can upload and create textual instructions, video and/or audio instructions, photos, pictures, etc. to be provided to the trainee while the trainee is participating in a training session for each Task Step of each Task with each Module of the Program.

For example, for each Task Step, the user can create and assign a number of fields to be associated with the Task Step, as shown in the following Table 2:

TABLE 2

| Field | Function |
|---|---|
| Task Step Name | Names the task step so that it can |
| | become searchable. |
| Task Step Content | Text to be displayed on the screen for |
| | this step when the trainee uses the |
| | program. |
| Next Step Text | Text to be displayed on the task step |
| | near the button the trainee will press to |
| | advance to the next step in the task. |
| Allow Alternate Step | Enables the user to program an |
| | alternate step so that a trainee can |
| | choose to follow a secondary set of |
| | steps. This is valuable when a trainee |
| | must make a decision during the task |
| | that may alter the next step that is |
| | needed. |
| Image for Alternate Step | Designated image to be displayed for |
| | the “Alternate Step” |
| Audio Cue | Designated audio cue to include on the |
| | task step. |
| Visual Cue | Designated visual cue, such as a static |
| | picture or a video clip. |
| Add a Subtask | Allows a user to select from a menu of |
| | subtasks that can be included on this |
| | task step when the trainee accesses it. |
| | This is core to the “problem solving” |
| | feature in the program. |

Once all of the information has been entered, the user can view a preview of how the prompts for the Task Step will appear to the trainee when accessed during a training session. The user can then edit and revise the fields for the Task Step, as necessary.

In this way, the systems and methods of the present disclosure enable a trainer, such as a direct service provider or teacher, to provide training during training session to a trainee for various Programs, Modules, Tasks, and Task Steps, while tracking the trainee's performance and progress. The system enables the trainer to track and evaluate individual Task Steps and provide a classification for the level of independence exhibited by the trainee in performing the Task Step. The system also provides a recommendation for a complexity level for the amount of guidance to be provided by the trainer the next time the trainee performs the same Task based on the previous evaluation of the Tasks Steps.

The foregoing description of the embodiments has been provided for purposes of illustration and description and is not intended to be exhaustive or to limit the disclosure. Individual elements or features of a particular embodiment are generally not limited to that particular embodiment, but, where applicable, are interchangeable and can be used in another embodiment, even if not specifically shown or described. The various embodiments may also be varied in many ways. Such variations are not to be regarded as a departure from the disclosure, and all such modifications are intended to be included within the scope of the disclosure. Although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Example embodiments are provided so that this disclosure will be thorough and will fully convey the scope to those who are skilled in the art. Specific details are set forth, including examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

In the written description and claims, one or more steps within a method may be executed in a different order (or concurrently) without altering the principles of the present disclosure. Similarly, one or more instructions stored in a non-transitory computer-readable medium may be executed in different order (or concurrently) without altering the principles of the present disclosure. Unless indicated otherwise, numbering or other labeling of instructions or method steps is done for convenient reference and not to indicate a fixed order.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

The phrase “at least one of A, B, and C” should be construed to mean a logical (A OR B OR C), using a non-exclusive logical OR, and should not be construed to mean “at least one of A, at least one of B, and at least one of C.” The term “set” does not necessarily exclude the empty set. The term “non-empty set” may be used to indicate exclusion of the empty set. The term “subset” does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information, but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “server” or “computing device” may refer to, be part of, or include processor hardware (shared, dedicated, or group) that executes code and memory hardware (shared, dedicated, or group) that stores code executed by the processor hardware.

The server or computing device may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard (including Core Specification versions 3.0, 4.0, 4.1, 4.2, 5.0, and 5.1 from the Bluetooth SIG) from the Bluetooth Special Interest Group (SIG).

The server or computing device may communicate with other servers or computing devices using the interface circuit(s). Although the server or computing device may be depicted in the present disclosure as logically communicating directly with other servers or computing devices, in various implementations the servers or computing devices may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the server or computing device may be distributed among multiple servers or computing devices that are connected via the communications system. For example, multiple servers or computing devices may implement the same functionality distributed by a load balancing system. In a further example, the functionality of the server and/or computing device be split between a remote or cloud server and a client computing device. For example, the client computing device may include a native application executing on a client computing device and in network communication with the server.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media.

The apparatuses and methods described in this application may be partially or fully implemented by a special purpose computer created by configuring a general-purpose computer to execute one or more particular functions embodied in computer programs. Such apparatuses and methods may be described as computerized apparatuses and computerized methods. The functional blocks and flowchart elements described above serve as software specifications, which can be translated into computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A system comprising:
a trainer computing device configured for operation by a trainer providing training to a trainee, the trainee having a developmental disability, the trainer computing device being configured for communication with a server that stores data for at least one task having a plurality of task steps, each task step having associated instructions for performing the task step;
a trainee computing device configured for operation by the trainee, the training computing device being configured for communication with the server and configured to output the associated instructions for each task step of the plurality of task steps while the trainee is performing the at least one task;
wherein:
the trainer computing device enables the trainer to: monitor progress of the trainee operating the trainee computing device while the trainee is performing the plurality of task steps of the at least one task; and assign a task step score from a plurality of task step scores for each task step of the plurality of task steps after the trainee has completed each task step, the task step score indicating a level of guidance provided by the trainer to the trainee while the trainee was completing each task step; and
the server is configured to select a complexity level from a plurality of complexity levels to be used by the trainer during a subsequent training session when the trainee performs the at least one task based on the task step scores for the plurality of task steps, the complexity level indicating training conditions and an amount of oversight to be provided by the trainer to the trainee during the subsequent training session.

2. The system of claim 1, wherein the associated instructions for each of the plurality of tasks steps output by the trainee computing device to the trainee while the trainee is performing the at least one task include at least one of: video output; audio output; image output; and textual output.

3. The system of claim 1, wherein the plurality of task step scores include an independent score indicating that the trainee was able to independently complete the associated task step without assistance.

4. The system of claim 3, wherein the server is configured to select the complexity level based on a number of task steps from the plurality of task steps that were assigned with the independent score by the trainer.

5. The system of claim 3, wherein the plurality of task step scores additionally include at least one of: a full physical score indicating that the trainer provided full physical assistance to the trainee while the trainee was performing the associated task step; a partial physical score indicating that the trainer provided partial physical assistance to the trainee while the trainee was performing the associated task step; and a gesture score indicating that the trainer provided a gesture to the trainee to assist the trainee while the trainee was performing the associated task step.

6. The system of claim 1, wherein the trainer computing device is configured to display the associated instructions that are also being output to the trainee with the trainee computing device.

7. The system of claim 1, wherein the trainer computing device is configured to direct the trainee computing device, through communication with the server, to move to a different task step within the plurality of task steps based on input received from the trainer with the trainer computing device.

8. The system of claim 1, wherein each of the plurality of complexity levels indicate at least one of: a trainer-to-trainee ratio; whether the trainer is in close proximity to the trainee; whether the trainer is in a room with the trainee; and whether verbal praise is provided after each step.

9. The system of claim 1, wherein the trainee computing device is configured to provide additional instructions to the trainee in response to the trainee encountering a problem during performance of a task step.

10. The system of claim 1, wherein the trainer computing device and the trainee computing device are each one of a laptop, a tablet, and a smartphone.

11. A method comprising:
communicating, using a trainer computing device, with a server that stores data for at least one task having a plurality of task steps, each task step having associated instructions for performing the task step, the trainer computing device being configured for operation by a trainer providing training to a trainee, the trainee having a developmental disability;
communicating, using a trainee computing device, with the server, the trainee computing device being configured for operation by the trainee and configured to output the associated instructions for each task step of the plurality of task steps while the trainee is performing the at least one task;
monitoring, with the trainer computing device, progress of the trainee operating the trainee computing device while the trainee is performing the plurality of task steps of the at least one task;
assigning, with the trainer computing device, a task step score from a plurality of task step scores for each task step of the plurality of task steps after the trainee has completed each task step, the task step score indicating a level of guidance provided by the trainer to the trainee while the trainee was completing each task step;
determining, with the server, a complexity level from a plurality of complexity levels to be used by the trainer during a subsequent training session when the trainee performs the at least one task based on the task step scores for the plurality of task steps, the complexity level indicating training conditions and an amount of oversight to be provided by the trainer to the trainee during the subsequent training session.

12. The method of claim 11, wherein the associated instructions for each of the plurality of tasks steps output by the trainee computing device to the trainee while the trainee is performing the at least one task include at least one of: video output; audio output; image output; and textual output.

13. The method of claim 11, wherein the plurality of task step scores include an independent score indicating that the trainee was able to independently complete the associated task step without assistance.

14. The method of claim 13, wherein the server is configured to determine the complexity level based on a number of task steps from the plurality of task steps that were assigned with the independent score by the trainer.

15. The method of claim 13, wherein the plurality of task step scores additionally include at least one of: a full physical score indicating that the trainer provided full physical assistance to the trainee while the trainee was performing the associated task step; a partial physical score indicating that the trainer provided partial physical assistance to the trainee while the trainee was performing the associated task step; and a gesture score indicating that the trainer provided a gesture to the trainee to assist the trainee while the trainee was performing the associated task step.

16. The method of claim 11, further comprising displaying, with the trainer computing device, the associated instructions that are also being output to the trainee with the trainee computing device.

17. The method of claim 11, further comprising directing, with the trainer computing device, the trainee computing device, through communication with the server, to move to a different task step within the plurality of task steps based on input received from the trainer with the trainer computing device.

18. The method of claim 11, wherein each of the plurality of complexity levels indicate at least one of: a trainer-to-trainee ratio; whether the trainer is in close proximity to the trainee; whether the trainer is in a room with the trainee; and whether verbal praise is provided after each step.

19. The method of claim 11, further comprising providing, with the trainee computing device, additional instructions to the trainee in response to the trainee encountering a problem during performance of a task step.

20. The method of claim 11, wherein the trainer computing device and the trainee computing device are each one of a laptop, a tablet, and a smartphone.
